(19) United States
(12) Patent Application Publication
(10) Pub. No.: US 2013/0317404 A1
(43) Pub. Date: Nov. 28, 2013

(54) ANKLE JOINT ORTHOSIS

(71) Applicant: medi GmbH & Co. KG, Bayreuth (DE)

(72) Inventors: Patrick BAUER, Auerbach (DE); Wolfgang KUBRICH, Lichtenfels (DE); Peter HOFFEINS, Bayreuth (DE)

(21) Appl. No.: 13/900,925

(22) Filed: May 23, 2013

(30) Foreign Application Priority Data
May 24, 2012 (DE) 102012010240.2

(51) Int. Cl. A61F 5/01

(52) U.S. Cl. CPC A61F 5/0111
USPC 602/27

(57) ABSTRACT

The ankle joint orthosis immobilizes the ankle joint. The orthosis has an outer knitted part and an inner one-piece stabilizing element. The knitted part is knitted in a shape adapted to the anatomy of the area of the ankle joint and completely encloses the ankle joint. The knitted part opens in order to fit it in the position of use and then drawn together and tightened. The one-piece stabilizing element has a heel portion, a side portion which adjoins the heel portion and extends along the side of the ankle joint, and a calf portion which adjoins the side portion and engages around the rear of the calf. One or more first connector elements provided on the outside of the stabilizing element are releasably connecting to one or more second connector elements provided on the inside of the knitted part.

The invention relates to an ankle joint orthosis, designed to immobilize the ankle joint.

Huge numbers of ankle joint injuries occur each day. For conservative treatment, an ankle joint orthosis is often used, which serves to stabilize and immobilize the foot in such a way that the foot cannot buckle sideways and cannot be flexed upward and downward. Known orthoses are usually composed of a textile sheet material which is knitted in a shape adapted to the anatomy of the leg and foot in the area of the ankle joint. This textile sheet material, in the position of use, completely encloses the ankle joint and the adjoining areas of the leg and foot. In order to fit the orthosis in place, it can be widened or opened such that the patient is able to step into it almost like stepping into a shoe. The textile sheet material is then drawn together by suitable tightening means, usually a shoelace, which is suitably guided and pulled tight, such that the textile sheet material bears firmly around the area of the ankle joint. Suitably firm tightening provides stable engagement of the ankle, and this also compresses any swelling that may occur.

For further stabilization against lateral buckling, it is also known to place one or more stabilizing elements on the outside of the textile sheet material and to fix them in position using one or more straps which are to be wound in a predetermined and defined configuration around the textile sheet material and the one or more stabilizing elements. Although this permits even better lateral stabilization, applying the one or more stabilizing elements and fixing them in position and binding the one or more straps is an awkward thing to do, especially for people who, for example because of their age or injuries, find it difficult to reach their ankles.

The problem addressed by the invention is therefore to make available an ankle joint orthosis which, firstly, has very good lateral stabilization and, secondly, is of simple design, such that it can easily be fitted in place without great effort.

To solve this problem, an ankle joint orthosis according to the invention is provided with a knitted part which is knitted in a shape adapted to the anatomy of the leg and foot in the area of the ankle joint and which, in the position of use, completely encloses the ankle joint and the adjoining areas of the leg and foot, and which can be opened or widened in some areas in order to fit it in place and, in the position of use, can be drawn together and tightened by tightening means, and also a one-piece stabilizing element with a heel portion receiving the heel in the position of use, a side portion which adjoins the heel portion and extends along the side of the ankle joint, and a calf portion which adjoins the side portion and engages around the rear of the calf, wherein one or more first connector elements are provided on the outside of the stabilizing element, and one or more second connector elements are provided on the inside of the knitted part, in such a way that the stabilizing element, insertable into the knitted part, can be fixed releasably in the knitted part by connection of the one or more first connector elements to the one or more second connector elements.

The ankle joint orthosis according to the invention is designed in two parts, consisting of the knitted part and the stabilizing element. The stabilizing element, which is preferably a structural part made of plastic, has a defined shape consisting of three distinct portions, namely a heel portion, which is arranged under the heel in the position of use and engages laterally around the heel, a side portion which adjoins the heel portion and extends upward along the ankle joint to the calf, and a calf portion which adjoins the side portion and engages like a half-shell around the calf. This stabilizing element provides stiffening and even better protection against lateral buckling than is possible using the knitted part on its own.

This stabilizing element is not applied to the outside of the knitted part where (see above description) it then has to be fixed in a complicated and awkward way. Instead, according to the invention, this stabilizing element is inserted into the already anatomically pre-shaped knitted part and is fixed releasably there via connector elements provided on the stabilizing element and on the knitted part. This allows the stabilizing element to be inserted into the knitted part before the latter is put on. The correct positioning of the knitted part is very easy, since the first and second connector elements are arranged at corresponding locations, which are chosen such that the stabilizing element, in the defined connection location, is automatically positioned in an optimal manner in the knitted part. In another location, the stabilizing element cannot be fixed in the knitted part. Once the patient has inserted the stabilizing element into the knitted part, he can step easily into the orthosis, slipping the foot into the stabilizing element and also into the knitted part. After the orthosis has been put on, the knitted part simply has to be pulled tight via the tightening means, i.e. the shoelace or the like. Once this has been done, the ankle joint orthosis is finally in place. Since the stabilizing element has been placed in the correct and optimal position in the knitted part before the latter is put on, the foot is likewise received optimally both in the stabilizing element and also in the knitted part. Any other complicated maneuvering for fixing the stabilizing element, as is needed in the prior art, is no longer needed in the orthosis according to the invention. Moreover, the way in which the ankle joint orthosis is put on is basically self-explanatory since, as has already been stated, all that needs to be done is to arrange the stabilizing element in the knitted part using the connector elements, after which the orthosis simply has to be put on and laced up. There is no need to learn any complicated configurations of a strap or the like.

The first and second connector elements, via which the stabilizing element and the knitted part are releasably connected, are preferably provided in the form of hook-and-loop fasteners. These can be provided on the stabilizing element, for example on the outside of the calf portion, preferably centrally, and on the outside of the heel portion, preferably centrally. The corresponding second hook-and-loop fasteners are provided at corresponding locations on the knitted part lying opposite in the position of use. Alternatively, press-stud fasteners or the like can of course also be provided.

As has been described, the stabilizing element is preferably made of plastic. Since it is releasable, it can be very easily removed from the knitted part for cleaning purposes. As a structural part made of plastic, it is also easy to clean.

The first connector elements on the stabilizing element, in particular the hook-and-loop fasteners, can be affixed to the outside of the stabilizing element, although it is also conceivable for them to be injection-moulded directly into the stabilizing element made of plastic, i.e. to be cohesively connected directly to the stabilizing element.

According to a particularly advantageous development, provision is made that the stabilizing element has different degrees of stiffness in some sections, in such a way that the side portion is harder than at least parts of the calf portion and of the heel portion. The central function of the stabilizing element is lateral stabilization, which prevents lateral buckling. For this purpose, it is advantageous for the side portion to be made relatively hard. For good wearing comfort, however, it is advantageous that those areas in particular which are loaded during use or which are brought into firm contact with the body during lacing, namely areas of the heel portion and also of the calf portion, are made relatively soft, such that they are flexible and adaptable, i.e. are able to easily adapt to the shape of the foot and heel area. This is achieved by virtue of the fact that the stabilizing element made of plastic is made softer specifically in these sensitive areas than in the side portion. Moreover, the heel portion, in a continuation of the side portion, can be equally as hard as the side portion, i.e. the hard side portion is as it were extended slightly into the heel portion, preferably to a point under the patient's heel, whereas the other areas of the heel portion are softer, in particular those areas laterally enclosing the heel. The fact that the stabilizing element is made of plastic permits any desired adaptation or adjustment of the softness, by using different plastics, or plastics of the same type but with a different Shore hardness, to form the stabilizing element. Production is easily possible by multi-component injection molding.

In a preferred development of the invention, provision is made that the knitted part is composed of a knitted portion and of a tongue, which is made harder than the knitted portion particularly by means of a shell-shaped insert part, wherein the tongue is secured on the knitted portion in such a way that it can be folded outward. The knitted part is thus composed of a sufficiently rigid knitted portion, which is knitted in such a way that it is adapted to the shape of the foot in the area engaged. It is open in the area of the shin and upper foot, where the knitted portion is drawn over the stabilizing element for fixing. A tongue is secured on the knitted portion and can be folded outward, such that the knitted part can thus be opened wide, which makes it very easy to step into the orthosis even with an integrated stabilizing element. After the foot has been placed in the orthosis, the tongue part is placed against the leg, i.e. pivoted back into the knitted portion, and the knitted portion engages with its longitudinal sides around the tongue, over which the shoelace is guided. Once the latter has been pulled tight, the edges of the knitted portion are drawn firmly together over the tongue.

For reasons of stabilization, it is particularly advantageous if the tongue is made hard, which is easily possible by means of a shell-shaped insert part. Of course, suitable padding is provided on the inner face of the tongue. The knitted part itself is a rigid knit, which is knitted as far as possible without seams and is anatomically adapted. The tensile strength and rigidity of the knit are preferably achieved by means of twill constructions and a weft insertion. The elasticity, needed in the heel area in order to ensure that the knitted part can adapt to the shape of the heel in this area, is preferably achieved by a rib construction with partial weft insertion, preferably designed with elastic knitting and weft yarn materials. Other knitting techniques, knitting materials and weft yarn materials are conceivable and well known. The shape of the knit is also preferably achieved using what is called the gusset technique, which is employed without the need for further make-up and stitching.

As has been described, the tightening means provided is preferably in the form of a shoelace, with which the knitted portion can be drawn together over the tongue. For this purpose, at the edges of the knitted portion, ghilly eyelets are expediently provided in the foot area and hooks are provided in the leg area, and the shoelace is guided through these eyelets and around these hooks in order to provide tightening. The shoelace can have an integrated damp system, which allows the shoelace, after it has been pulled tight, to be easily damped without the need to tie a loop.

The direct integration of the stabilizing element, which as has been stated bears directly on the skin of the leg and the joint area, in conjunction with the external fixing and bracing via the knitted part and tongue, already leads to excellent immobilization of the ankle joint. By virtue of the stabilizing element being arranged on the inside and, consequently, being in direct contact in the area to be stabilized, it is possible to achieve a direct stabilizing effect, in contrast to previously known orthoses in which the stablilizing effect of the stabilizing element is achieved only indirectly, since a textile sheet material is always arranged between the stabilizing element and the area to be stabilized. There, an indirect stabilization is therefore always obtainable. However, in the orthosis according to the invention, in order to still further improve the already excellent stabilization, which can usually be the case only in particular problematic cases arising from the nature of the injury, it is possible according to the invention to further provide a strap, at the two ends of which there are third connector elements which can be releasably connected to fourth connector elements provided on the outside of the knitted part, wherein the strap is guided around the knitted part in a figure-of-eight configuration, so as to provide further immobilization in the position of use. By means of this strap, which serves exclusively for immobilization and not for the fixing of a stabilizing element or the like, almost complete immobilization of the ankle joint can be achieved. If necessary, the strap can therefore be used to upgrade the orthosis, for example when swelling has subsided after application of the “basic orthosis” consisting of stabilizing element and knitted part, but when complete immobilization is temporarily needed thereafter. After a certain period of treatment, the strap can be removed, and the stabilization afforded by the “basic orthosis” may be sufficient. Toward the end of the treatment, it is then also possible to do without the stabilizing element, such that the ankle joint is fixed only by the knitted part.

As has been described, the third connector elements are provided on the strap, at the ends thereof, and the fourth connector elements are provided on the knitted part, at the side areas thereof to which the strap ends are guided in the figure-of-eight configuration. However, in order to fix the strap, it is also conceivable, before it is actually applied, to provide a further third and fourth connector element at the center of the strap and on the underside of the heel area of the knitted part.

These connector elements too are preferably designed as hook-and-loop fasteners, although press-stud connectors or the like are also conceivable.

Further advantages, features and details of the invention will become clear from the illustrative embodiment described below and from the drawing, in which:

FIG. 1 shows a perspective view of a knitted part of an ankle joint orthosis according to the invention,

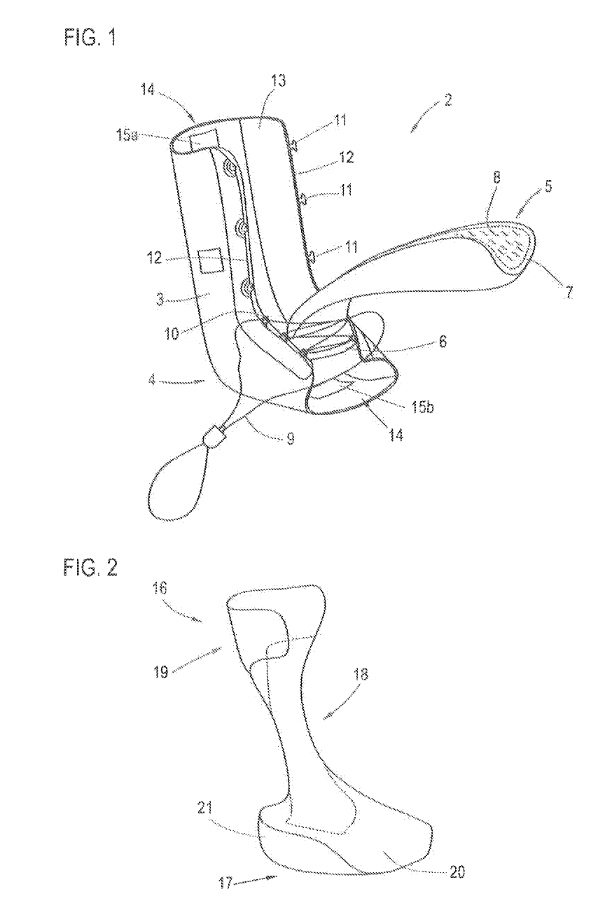

FIG. 2 shows a perspective view of a stabilizing element of the orthosis according to the invention,

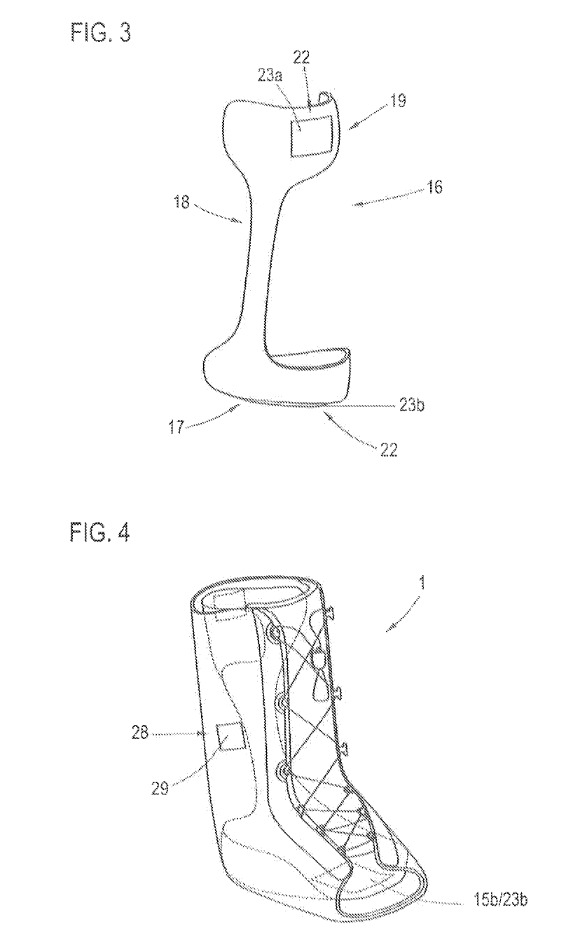

FIG. 3 shows the stabilizing element from FIG. 2 in a rear view,

FIG. 4 shows the ankle orthosis according to the invention with the stabilizing element inserted into the knitted part, and

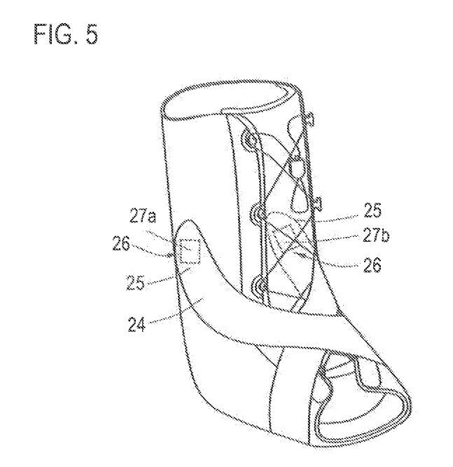

FIG. 5 shows the ankle joint orthosis from FIG. 4 with an additionally applied strap.

FIG. 1 shows, as part of an ankle joint orthosis 1 according to the invention and as shown in FIG. 4, a knitted part 2 composed of a knitted portion 3, which is knitted as a seamless, rigid knit in such a way that it is adapted to the anatomy of the foot. Its tensile strength and its rigidity are achieved by means of twill constructions and a weft insertion. In the heel area 4, it is knitted elastically, which is preferably achieved by a rib construction with partial weft insertion. Elastic knitting and weft yarn materials are preferably used. The anatomically adapted shape of the knit is preferably achieved by the gusset technique.

A tongue 5 is arranged movably on the knitted portion 3, for which purpose corresponding elastic band connections 6 are provided, which are secured on the knitted part 3 and also on the tongue 5, preferably being sewn on. The tongue 5 is thus connected nonreleasably to the knitted portion 3. It has a sufficiently stable insert 7 (shown only by broken lines here), which has the shape of a half shell and serves to stiffen the tongue. The tongue is therefore very stable in itself and is sufficiently stiffened by the insert 7, for example a suitable shell made of plastic. Padding 8 is provided on the inner face of the tongue 5 in order to permit a cushioned fit on the patient's leg.

A tightening means is also provided, here in the form of a shoelace 9 which is guided through a series of ghilly eyelets 10, such that it cannot very easily come undone. For final tightening and lacing, it is then guided around several hooks 11, which are secured on the edges 12 of the knitted part 3 (as also are the ghilly eyelets), after which it is pulled tight and laced up for fixing.

In the example shown, several first connector elements 14 are securely arranged at different positions on the inside 13 of the knitted portion 3. These first connector elements are preferably hook-and-loop fasteners. A first hook-and-loop fastener 15a is located in the upper calf area, and a second hook-and-loop fastener 15b is located in the heel or foot area of the knitted portion 3. These hook-and-loop fasteners 15a, 15b serve to secure and fix a stabilizing element, which is discussed in more detail below. The hook-and-loop fasteners are securely sewn or adhesively bonded onto the knitted portion 3. As an alternative to the use of hook-and-loop fasteners, it would also be possible in principle to use press-studs or similar, although hook-and-loop fasteners in particular are especially easy to secure and also to connect.

FIGS. 2 and 3 show a stabilizing element 16, which is designed as a structural part made of plastic. It is composed of three portions, namely the heel portion 17, the side portion 18, and the calf portion 19. The heel portion 17 has a shell-shaped design, with a lower heel support area 20 merging into a side area 21 which engages round the rear and sides of the heel. It merges in one piece into the side portion 18, which extends upward from the foot portion 17 and, in the position of use, runs along the side of the damaged ankle joint. This side portion 18 in turn merges in one piece into the calf portion 19 which, in the position of use, engages like a half shell around the rear of the calf.

The side portion 18 is made harder than the calf portion 19 and also most of the heel portion 17. It is therefore stiffer and has a different Shore hardness than the other portions. Its purpose is exclusively to provide stabilization in order to avoid buckling of the ankle joint, which is why a high degree of stiffness is advantageous. However, as is indicated by broken lines in FIG. 2, the area of increased stiffness continues slightly into the heel portion 17 and into the area of the heel support 20, that is to say under the sole and the heel. By contrast, all the other portions of the stabilizing element 16 are substantially softer and more flexible, such that they are readily able to adapt and conform to the shape of the foot and calf, respectively, and therefore, in these areas, there are no places where pressure or rubbing occurs as a result of excessively stiff material.

As can be seen from the rear view in FIG. 3, first connector elements 22 are provided on the calf portion 19, on the side portion 18 and on the heel portion 17 and are designed to be congruent with respect to the connector element of the knitted portion 3. Since hook-and-loop fasteners 15a, 15b are preferably used there, the first connector elements 22 are therefore likewise designed as hook-and-loop fasteners 23a, 23b. They are arranged on the stabilizing element 16 at positions which are chosen such that the stabilizing element 16, when inserted into the knitted part 2, is automatically positioned in an optimal manner in the knitted part, which is made possible by the connection of the hook-and-loop fasteners 15a/23a and 15b/23b.

The first connector elements 22, i.e, the hook-and-loop fasteners 23a, 23b, are preferably injection-molded directly into the plastics material of the stabilizing element 16, so as to give a cohesive material connection. However, it is also conceivable for them simply to be affixed.

To put on the ankle joint orthosis 1, it is simply a matter of inserting the stabilizing element 16 into the knitted part 2. For this purpose, the knitted part 2 is easily opened, by means of the tongue 5 being folded outward, as is shown by way of example in FIG. 1. The stabilizing element 16 is then inserted into the knitted part 2 and moved and positioned until the hook-and-loop fasteners 15a/23a and 15b/23b lie opposite each other and can be connected to each other. After these have been connected, the stabilizing element 16 is fixed firmly and in a precise position in the knitted part 2 or in the knitted portion 3.

The patient then steps into the still open orthosis. In doing so, the foot is automatically guided into the stabilizing element 16. The foot sits with its heel on the heel support area 20 and in the heel portion 17, and the ankle joint lies directly adjacent to the side portion 18, while the calf is engaged by the calf portion 19. For fixing and immobilization, all that now needs to be done is to swivel the tongue 5 inward such that the padding 8 bears on the shin and the upper foot. The side edges 12 of the knitted portion 3 lie on the outside of the tongue 5. All that remains to be done now is for the shoelace 9 to be suitably guided and pulled tight and laced up, such that the edges 12 of the knitted portion are pulled firmly toward each other, whereby the rigid knit is automatically drawn tightly around the joint area. Here, the tongue 5 is likewise pressed with its padding 8 against the joint area. However, on account of the rigidity of the knitted portion 3, the stabilizing element 16 is thus also secured relative to the joint area or foot, and the foot is safely protected by the stabilizing element 16 against lateral buckling. Depending on the design of the tongue, however, this does not necessarily rule out an upward and downward movement.

In order to achieve still further immobilization also with respect to this movement, a strap 24 is provided (see FIG. 5), at the two ends 25 of which there are third connector elements 26, preferably again in the form of hook-and-loop fasteners 27a, 27b. Corresponding fourth connector elements 28 are provided (only one is shown in FIG. 4) on the outside of the knitted portion 3, in the region of the side areas, and are once again designed as hook-and-loop fasteners 29. There can also be a third hook-and-loop fastener at the center of the strap, and, correspondingly, there is also a fourth hook-and-loop fastener on the underside of the knitted portion 3, such that the strap can also be fixed at the center.

As FIG. 5 shows, the strap 24 is now arranged in a figure-of-eight configuration and tightened around the already fitted ankle joint orthosis 1. Since the strap is stable in itself, i.e. does not lengthen, further immobilization can be achieved by pulling it suitably tight.

It will be appreciated that the system, comprising a maximum of three parts, is fully flexible to the extent that, depending on the type of injury or the state of healing, it is possible to apply either all three elements, only the knitted part 2 and the stabilizing element 16, or only the knitted part 2. For example, immediately after an injury has been sustained with resulting swelling, it is possible to apply only the knitted part 2 with an inserted stabilizing element 16. After the swelling has subsided, the strap 24 can additionally be applied. As healing progresses, the strap 24 can be removed, such that immobilization is then only provided by the knitted part 2 and the stabilizing element 16. As the healing process continues still further, the stabilizing element can then be removed, and the stabilization is then provided only by the knitted part 2.

1. Ankle joint orthosis designed to immobilize the ankle joint, comprising a knitted part which is knitted in a shape adapted to the anatomy of the leg and foot in the area of the ankle joint and which, in the position of use, completely encloses the ankle joint and the adjoining areas of the leg and foot, and which can be opened or widened in some areas in order to fit it in place and, in the position of use, can be drawn together and tightened by tightening means, and also a one-piece stabilizing element with a heel portion receiving the heel in the position of use, a side portion which adjoins the heel portion and extends along the side of the ankle joint, and a calf portion which adjoins the side portion and engages around the rear of the calf, wherein one or more first connector elements are provided on the outside of the stabilizing element, and one or more second connector elements are provided on the inside of the knitted part, in such a way that the stabilizing element, insertable into the knitted part, can be fixed releasably in the knitted part by connection of the one or more first connector elements to the one or more second connector elements.

2. Ankle joint orthosis according to claim 1, wherein hook-and-loop or press-stud fasteners are provided as first and second connector elements.

3. Ankle joint orthosis according to claim 1, wherein a first connector element is provided on the calf portion, and a second connector element is provided on the calf area of the knitted part.

4. Ankle joint orthosis according to claim 1, wherein a first connector element is provided on the heel portion, and a second connector element is provided on the heel area of the knitted part.

5. Ankle joint orthosis according to claim 1, wherein the stabilizing element is made of plastic.

6. Ankle joint orthosis according to claim 5, wherein it has different degrees of stiffness in some sections, in such a way that the side portion is harder than at least parts of the calf portion and of the heel portion.

7. Ankle joint orthosis according to claim 6, wherein the heel portion, in a continuation of the side portion, is equally as hard as the side portion, said hard area extending into the lower area of the heel portion, whereas the other areas of the heel portion are softer.

8. Ankle joint orthosis according to claim 1, wherein the knitted part is composed of a knitted portion and of a tongue, which is made harder than the knitted portion particularly by means of a shell-shaped insert part, wherein the tongue is secured on the knitted portion in such a way that it can be folded outward.

9. Ankle joint orthosis according to claim 8, wherein the tightening means provided is in the form of a shoelace, with which the knitted portion can be drawn together over the tongue.

10. Ankle joint orthosis according to claim 9, wherein at the edges of the knitted portion, ghilly eyelets are provided in the foot area and hooks are provided in the leg area, and the shoelace is guided through these eyelets and around these hooks in order to provide tightening.

11. Ankle joint orthosis according to claim 8, wherein padding is provided on the inner face of the tongue.

12. Ankle joint orthosis according to claim 1, wherein a strap is furthermore provided, at the two ends of which there are third connector elements which can be releasably connected to fourth connector elements provided on the outside of the knitted part, wherein the strap is guided around the knitted part in a figure-of-eight configuration, so as to provide further immobilization in the position of use.

13. Ankle joint orthosis according to claim 12, wherein a further third and fourth connector element is provided at the center of the strap and on the underside of the heel area of the knitted part.

14. Ankle joint orthosis according to claim 12, the third and fourth connector elements are hook-and-loop or press-stud fasteners.
